(19) United States
(12) Patent Application Publication
(10) Pub. No.: US 2006/0276744 A1
(43) Pub. Date: Dec. 7, 2006

(54) Configuration for drug delivery systems

(76) Inventor: Theodore J. Falk, Clarence, NY (US)

Correspondence Address: HODGSON RUSS LLP, ONE M & T PLAZA, SUITE 2000, BUFFALO, NY, 14203-2391, US

(21) Appl. No.: 11/438,588

(22) Filed: May 22, 2006

Related U.S. Application Data
(60) Provisional application No. 60/683,253, filed on May 20, 2005.

(51) Int. Cl. A61M 31/00; A61K 9/22

(52) U.S. Cl. 604/67; 604/890.1

(57) ABSTRACT

A configuration for a drug delivery system which offers significant advantages over presently available systems. The system provides redundant protection against overdelivery of drug, increased accuracy of delivery, reduced energy requirement, increased capability to clear a blocked catheter, and alarms signifying catheter blockage or valve leakage. The system comprises the series combination of a reservoir, low power electromagnetic pump having spring-biased magnetically actuated piston/plunger and a check valve, pressure sensor, pressure regulator and a catheter. The reservoir supplies a drug at greater than ambient pressure, e.g. 18.7 psia or 4 psi above normal sea level ambient pressure. The pump increases the pressure by 3 psi to 21.7 psia. The pressure sensor tracks the pressure at this location and triggers suitable action by the pump. The pressure regulator downstream of the pressure sensor is configured to hold the pressure at the regulator inlet at a desired level such as 21.7 psia. The catheter at the regulator exit delivers fluid to the patient at a pressure which is normally close to that of the patient's environment.

### CROSS REFERENCE TO A RELATED APPLICATION

Applicant claims priority based on U.S. provisional patent application No. 60/683,253 filed May 20, 2005 entitled “Configuration For A Drug Delivery System” which is incorporated herein by reference.

### FIELD OF THE INVENTION

This invention relates to implantable systems for delivering liquids such as medicines, drugs, insulin, chemotherapy liquids and other life critical drugs to a patient.

### BACKGROUND OF THE INVENTION

An acceptable configuration for an implantable drug delivery system must be safe against overdelivery of drug. In most applications overdelivery is considered to be more dangerous than underdelivery. One prior art system configuration meets this requirement by employing a reservoir that stores drug at low pressure which is less than ambient pressure. Thus a leak along the flow path through the pump draws drug back into the reservoir, not into the patient. The reservoir of another prior art system stores drug at a pressure which is greater than ambient pressure (which may range under normal conditions from 8.7 psia at moderately high altitude to 19.7 psia if the patient swims underwater). Leak prevention redundancy is provided in this system by a combination of sealing by a peristaltic rotor and an outlet check valve. Ideally the rotor and the check valve, each acting alone, should be capable of preventing drug flow from the reservoir through the catheter. Overfilling of the reservoir is prevented by a valve in the fill system which closes when the reservoir is full.

The drug delivery must also be accurate. The accuracy of the pump in delivery systems can be adversely affected if bubbles are present in the flow. Some electromagnetic piston pumps for use in drug delivery systems are designed to continue pumping while a bubble is passing through it. However, passage of a bubble reduces the delivery rate of drug while the bubble is passing through the pump (assuming that the reservoir pressure is below delivery pressure) and it reduces the drug delivery rate to zero as the bubble exits the catheter.

In a system having a reservoir at constant pressure, bubbles may be prevented from passing through the pump by using a positive pressure reservoir, by degassing the drug prior to filling the reservoir, or by attempting to prevent the bubbles from leaving the reservoir by a suitable choice of an outlet filter. Refilling a positive pressure reservoir must be done very carefully or a safety system must be included to ensure that the reservoir is not overfilled thus causing overdelivery of drug. Degassing is a time-consuming process requiring some skill by the operator. The effectiveness of filters in preventing bubbles from passing through the pump has not yet been demonstrated.

A prior art system configuration incorporates in series a pressure regulator, an electromagnetic piston pump, a combined accumulator and pressure sensor, and a catheter. The pressure regulator serves the purpose of ensuring that an overfilled reservoir will not result in overdelivery of drug before the reservoir pressure decreases to the design value. The pressure sensor detects possible catheter blockage, the principal reason for pump failure, and also provides compliance in the flow path to act as an accumulator for the pump. Since bubbles will exist in the reservoir it is important that the volume of air reaching the pump and then the patient be limited. Bubble flow from the reservoir into the remainder of the flow system is to be prevented by a suitable choice of filter at the reservoir outlet.

### SUMMARY OF THE INVENTION

The invention provides a configuration for a drug delivery system which offers significant advantages over presently available systems. The system of the invention provides redundant protection against overdelivery of drug, increased accuracy of delivery, reduced energy requirement, increased capability to clear a blocked catheter, and alarms signifying catheter blockage or valve leakage. The system of the invention comprises the series combination of a reservoir, low power electromagnetic pump having spring-biased magnetically actuated piston/plunger and a check valve, pressure sensor, pressure regulator and a catheter. The reservoir supplies a drug at greater than ambient pressure, e.g. 18.7 psia or 4 psi above normal sea level ambient pressure. The pump increases the pressure by 3 psi to 21.7 psia. The pressure sensor tracks the pressure at this location and triggers suitable action by the pump. The pressure regulator downstream of the pressure sensor is configured to hold the pressure at the regulator inlet at a desired level such as 21.7 psia. The catheter at the regulator exit delivers fluid to the patient at a pressure which is normally close to that of the patient's environment.

The foregoing and additional advantages and characterizing features of the invention will become clearly apparent upon a reading of the ensuing detailed description together with the included drawings.

### BRIEF DESCRIPTION OF THE DRAWINGS

FIG. 1 is a schematic block diagram of the improved configuration for a drug delivery system according to the invention.

### DETAILED DESCRIPTION OF THE INVENTION

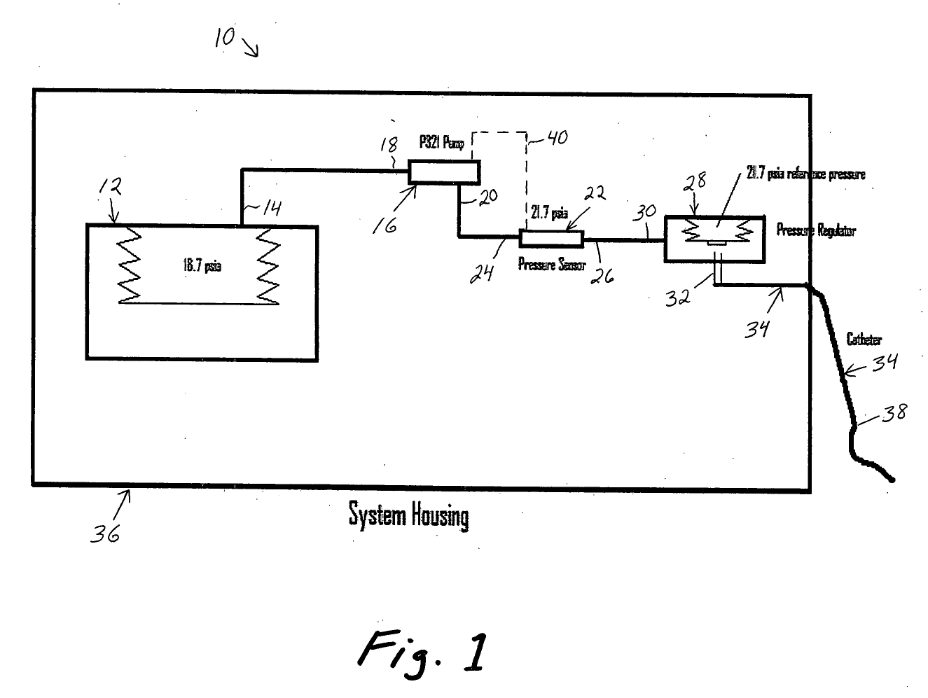

Referring to FIG. 1, the system 10 of the invention comprises a reservoir 12 for storing drug at a pressure greater than ambient pressure and having an outlet 14, a pump 16 having an inlet 18 in fluid communication with the outlet 14 of reservoir 12 and having an outlet 20, a pressure sensor 22 having an inlet 24 in fluid communication with outlet 20 of pump 18 and having an outlet 26, a pressure regulator 28 having an inlet 30 in fluid communication with outlet 26 of pressure sensor 22 and having an outlet 32 and a catheter 34 in fluid communication with outlet 32 of regulator 28. Thus, as shown in FIG. 1, reservoir 12, pump 16, pressure sensor 22, pressure regulator 28 and catheter 34 are in series flow relation, reservoir 12 being at the upstream end of the system 10 and catheter 34 being at the downstream end thereof.

For implantation of system 10 in the body of a patient, reservoir 12, pump 16, pressure sensor 22, pressure regulator 28 and a portion of the length of catheter 34 are contained within a housing 36 of a material suitable for implanting in the body of a patient. Such materials are well-known to those skilled in the art. As shown in FIG. 1, a portion of the length of catheter 34 is located within housing 36 and a remaining or external portion 38 extends to a location in the body of the patient to receive the drug.

Reservoir 12 can be of various commercially available forms readily known to those skilled in the art. Pump 16 is a low power electromagnetic pump having a spring-biased magnetically actuated piston/plunger and a check valve. One form of such pump found to perform satisfactorily in the system 10 of the invention is shown and described in the pending United States patent application filed May 19, 2006 under Express Mail Certificate of Mailing ER 952418865 US and entitled “Low Power Electromagnetic Pump”, the disclosure of which is hereby incorporated by reference. This pump is also referred to herein as the P321 pump. A pressure sensor 22 and a pressure regulator 28 each found to perform satisfactorily in the system 10 of the invention each is available from Medical Research Products of Valencia, Calif.

The reservoir 12 of this flow system 10 supplies drug at greater than ambient pressure. In this example the reservoir pressure is chosen to be 18.7 psia, that is 4 psi above normal sea level ambient pressure. This is followed by the pump 22 which increases the pressure by 3 psi to 21.7 psia. The pressure sensor 22 tracks the pressure at this location and triggers suitable action by the pump. The pressure regulator 28 downstream of the pressure sensor 22 is configured to hold the pressure at the regulator inlet 30 at the desired 21.7 psia. The catheter 34 at the regulator exit 32 delivers fluid to the patient at a pressure which is normally close to that of the patient's environment.

The system of the invention satisfies the requirement that there be redundant protection against leaks in the following manner. Protection against overdelivery involves providing protection in the event of regulator failure and protection in the event of pump failure. Turning first to regulator failure, if the regulator 28 fails completely then the pump 16 must prevent excessive flow through the system from the reservoir 12 at 18.7 psia to the end of the catheter 34 which may be at a pressure as low as 8.7 psia if the patient is at moderate altitude. The main check valve of the P321 pump 16 is capable of holding off a pressure of approximately 17 psid provided that the pump is not activated. If the pump 22 is activated the piston/plunger thereof will return to its rest position so long as the pressure drop across the piston/plunger is no greater than 11 psi. Thus no continuous flow should take place after a complete pressure regulator failure provided that the pressure at the delivery end of the catheter is no larger than 18.7−11=7.7 psia. This can only occur at high altitude. Some overdelivery will occur if the pressure at the pump outlet 20 decreases below 12.7 psia (with complete regulator failure) and the pump continues to cycle. Therefore the sensor 22 output, represented by line 40 in FIG. 1, should be used to shut down the pump 16 if the sensor pressure should decrease below 18.7 psia.

Turning next to pump failure, a complete failure of the pump 16 to seal would cause the regulator 28 to experience a pressure equal to 18.7 psia at its inlet port 30. The regulator should seal if the inlet pressure is less than 21.7 psi. Thus complete failure of either component should not result in extreme overdelivery of drug. If the pump 16 should fail because the piston/plunger thereof failed to return to its rest position at the end of a pump cycle, the pressure at the sensor would probably decrease below the 21.7 psia set value and a failure would be detected. If the pump should fail in a manner such that it continued to pump but did not deliver full stroke volume, this failure would not be detected. It is possible, however, that if the pressure sensor 22 is designed to have quick enough response, then analysis of the pressure waveform during the pump stroke may allow diagnosis of the nature of the failure.

Catheter clearing capability is provided by the configuration system of the invention in the following manner. The pressure sensor 22 should be capable of detecting pressures both above and below the desired inlet pressure to the pressure regulator 28 (in this case 21.7 psia). If the pressure regulator detects a pressure greater than 21.7 psia (in the present example) it indicates that the catheter is blocked. If the pump 16 is allowed to continue to pulse with normal excitation and with no flow through the catheter 34, the pressure at the regulator inlet 30 and in the catheter should increase to at least 38.7 psia before the pump stops. It should be noted that the P321 pump 16 is specified to be capable of a pressure increase of 20 psid with normal excitation. If upon detection of the blocked catheter, the duration of the exciting electrical pulse to the pump 16 is increased, a somewhat higher dead end pressure can be reached in an effort to clear the catheter 34. The designed 20 psid pressure capability of the pump unit 16 in the system 10 makes available 38.7 psia to clear the catheter 34 whereas a system with an 8.7 psia reservoir and the same pump can provide only 28.7 psia at the catheter inlet to clear the catheter.

The configuration system of the invention protects against reservoir depletion in the following manner. If the pressure sensor 22 detects a pressure less than the 21.7 psia normal value, then it indicates either a leak in the regulator 28, a failure of the pump 16, or depletion of the reservoir 12. If a moderate leak in the regulator causes the pressure at the sensor 22 to decrease below the set value, the system will still deliver the set flow rate of drug but the system 10 should be shut down because of the loss of redundant leak protection. If the regulator leak is severe enough to decrease the sensor pressure below 12.7 psia, then the pump 16 will begin to overdeliver and it certainly should be shut down. If the pressure in the reservoir decreases because the reservoir 12 is nearly empty then there may be no effect on the delivered flow until the pump 16 is no longer capable of increasing the pressure to 21.7 psia while delivering full stroke volume. Normally this would occur when the reservoir pressure decreased to 11.7 psia. However, the decrease in the pressure of the reservoir 12 would tend to cause the drug to outgas and the pump 16 might in that case stop delivering full stroke volume at a slightly higher pressure.

The configuration/system 10 of the invention has a number of additional advantages. One is improved accuracy. The configuration causes the pump 16 in a normally operating system to pump against a constant pressure independent of the delivery pressure. The delivery should therefore be very accurate. Another advantage is reduced energy requirement. Because the normal pressure increase across the pump 16 is low (3 psi in this example compared with 6 psi in a prior art design) the energy to drive the pump 16 can be reduced thus leading to a significant energy saving and increased pump life. Still another advantage is improved bubble pumping. The reduction in the pressure increase across the pump 16 will reduce the effect on delivered pulse volume caused by a bubble entering the pump. The accuracy of the pump 16 while passing bubbles will therefore be increased. It should be noted that a bubble event is less likely, however, because of the positive pressure in the reservoir 12.

While an embodiment of the invention has been described in detail, that has been for the purpose of illustration, not limitation.

1. A system for delivering liquid drug to a patient comprising:
a) a reservoir for storing drug at a pressure greater than ambient pressure and having an outlet;
b) a pump having an inlet in fluid communication with the outlet of the reservoir and having an outlet;
c) a pressure sensor having an inlet in fluid communication with the outlet of the pump and having an outlet;
d) a pressure regulator having an inlet in fluid communication with the outlet of the pressure sensor and having an outlet; and
e) a catheter in fluid communication with the outlet of the pressure regulator for providing drug to the patient.

2. The system according to claim 1, wherein the pump during normal operation thereof increases pressure at the outlet thereof to a level greater than the pressure of drug stored in the reservoir.

3. The system according to claim 1, wherein the pump comprises an electromagnetic pump having a spring-biased magnetically actuated piston/plunger and a check valve.

4. The system according to claim 1 wherein the pressure sensor is connected in controlling relation to the pump.

5. The system according to claim 1, wherein the regulator establishes and holds a system pressure at a level substantially equal to the pressure at the outlet of the pump.

6. The system according to claim 1, wherein the reservoir, pump, pressure sensor, pressure regulator and a portion of the catheter are contained within a housing of material suitable for implanting in the body of a patient, the catheter having a remaining portion for extending to a location in the body of the patient to receive the drug.

7. A system for delivering liquid drug to a patient comprising:
a) a reservoir for storing drug at a pressure greater than sea level ambient pressure and having an outlet;
b) a pump having an inlet in fluid communication with the outlet of the reservoir and having an outlet, the pump comprising an electromagnetic pump having a spring-biased magnetically actuated piston/plunger and a check valve, the pump during normal operation thereof increasing pressure at the outlet thereof to a level greater than the pressure of drug stored in the reservoir;
c) a pressure sensor having an inlet in fluid communication with the outlet of the pump and having an outlet, the pressure sensor being connected in controlling relation to the pump;
d) a pressure regulator having an inlet in fluid communication with the outlet of the sensor and having an outlet, the regulator establishing and maintaining a system pressure at a level substantially equal to the pressure at the outlet of the pump; and
e) a catheter in fluid communication with the outlet of the pressure regulator for providing drug to the patient.

8. The system according to claim 7, wherein the reservoir, pump, pressure sensor, pressure regulator and a portion of the catheter are contained within a housing of material suitable for implanting in the body of a patient, the catheter having a remaining portion for extending to a location in the body of the patient to receive the drug.

9. A method for operating a system to deliver liquid drug to a patient comprising:
a) providing a system comprising reservoir, pump, pressure sensor, pressure regulator and catheter in series flow relation;
b) storing drug in the reservoir at a pressure greater than ambient pressure;
c) pumping drug received from the reservoir to convey drug at a pressure greater than the pressure of drug stored in the reservoir;
d) sensing pressure of drug conveyed by the pump;
e) controlling the pump in response to sensed pressure;
f) utilizing the regulator to establish and maintain a system pressure at a level substantially equal to the pressure of the pumped drug; and
g) delivering drug to the patient via the catheter.

10. The method according to claim 9, further including containing the system in a housing of material suitable for implanting in the body of a patient.

11. The method according to claim 9, further including utilizing the pump to prevent excessive flow through the system in the event of failure of the regulator.

12. The method according to claim 11, wherein the pump comprises an electromagnetic pump having a spring-biased magnetically actuated piston/plunger and a check valve, and wherein the check valve is utilized to hold off pressure up to a predetermined magnitude when the pump is not activated.

13. The method according to claim 11, wherein the pump comprises an electromagnetic pump having a spring-biased magnetically actuated piston/plunger and a check valve, and wherein when the pump is activated the piston/plunger is returned to a rest position so long as the pressure drop across the piston/plunger is no greater than a predetermined magnitude.

14. The method according to claim 11, wherein the sensor is utilized to shut down the pump in response to system pressure decreasing below a predetermined magnitude.

15. The method according to claim 9, further including utilizing the regulator to prevent excessive flow through the system in the event of failure of the pump.

16. The method according to claim 9, wherein the pump comprises an electromagnetic pump having a spring-biased magnetically actuated piston/plunger and a check valve, and wherein the duration of the exciting electrical pulse to the pump is increased to provide an increased dead end pressure to clear a blockage in the catheter.

17. The method according to claim 9, further including utilizing the sensor to stop operation of the pump in the event of depletion of the reservoir.
